(19) United States
(12) Patent Application Publication
(10) Pub. No.: US 2007/0129609 A1
(43) Pub. Date: Jun. 7, 2007

(54) Support network system of medical institution

(76) Inventor: Kazuhiro Kawasaki, Hyogo (JP)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/581,508

(22) PCT Filed: Nov. 26, 2004
(86) PCT No.: PCT/JP04/17563

§ 371 Date: Nov. 2, 2006

(51) Int. Cl. A61B 5/00

(52) U.S. Cl. 600/300

(57) ABSTRACT

An objective of the present invention is to provide the latest medical treatment to a medical institution or a clinic in a remote/underpopulated area which is likely to lag behind the progress in the medical technology, which reduces economic burden on a terminal medical institution or a patient and establishes easier access for the patient to receive a close examination or diagnosis in the nearby medical institution, so that reducing labor or equipment burden on a specific latest medical institution or an expert while ensuring the accuracy in the diagnosis and examination. As a solving means thereof, the most-advanced diagnosis and examination are provided for the patient by arranging terminal equipments of essential diagnostic examination instruments in a plurality of the terminal medical institutions, such as the clinic in the remote/underpopulated area, performing diagnosis and examination of the patient by the terminal equipments at the terminal medical institution, transmitting diagnosis and examination data of the terminal equipment to a complete medical institution with specialized staff via a communication network, analyzing and processing the data at the complete medical institution, and transmitting the processed result to the terminal medical institution via the communication network.

### TECHNICAL FIELD

The present invention relates to a support network system of a medical institution, and relates to technology suitable for providing the latest medical treatment to a medical institution or a clinic in a remote/underpopulated area which is likely to lag behind the progress in the medical technology.

### BACKGROUND ART

Currently, medical instruments, medical devices, and medical equipments, as well as methods of medical treatment, have been progressing rapidly, and the latest diagnosis, examination, and treatment are performed in large-scale medical institutions or university hospitals by making full use of the latest medical instruments, medical devices, and medical equipments.

In contrast, in these large-scale medical institutions, it can happen that long waiting time is required and the actual medical examination takes only a few minutes.

On the other hand, in the medical institutions such as the clinic in a remote island or remote/underpopulated area, a problem arises that it makes difficult to receive the benefit of the latest medical technology due to such as the shortage of human resources such as highly-skilled physicians, or aging medical instruments, medical devices, and medical equipments, resulting in the necessity to go to urban areas to receive the latest medical treatment which requires a large amount of time transportation expenses.

As a solution to such a problem, it is considered to introduce the medical technology which involves, for example, transferring (transmitting) an image taken along a home diagnosis/examination or by the medical instrument, medical device, or medical equipment, to the large-scale medical institution in which a specialized physician resides to entrust diagnosis thereto using the currently-developed high-speed communication link;

Patent Document 1: Japanese Unexamined Patent Publication No. 2001-357134, “IMAGE PHOTOGRAPHING DEVICE AND IMAGE PROCESSOR”

Patent Document 2: Japanese Unexamined Patent Publication No. 2002-282250, “ULTRASONOGRAPHIC DEVICE”

Patent Document 3: Japanese Unexamined Patent Publication No. 2002-015062, “ADVANCED MEDICAL DIAGNOSTIC IMAGING SYSTEM”

Patent Document 4: Japanese Unexamined Patent Publication No. 2003-190098, “MEDICAL IMAGE PICKUP DATA STREAMING”

Patent Document 1 describes the technique where a patient himself/herself operates a device to take an image at home or the like and transfers the examination data to such as a support medical institution, which leaves problems that the accuracy of self-judgment by the patient is doubtful and the reliability is limited because the operation of the device is left to a seriously ill person or its caretaker.

Patent Document 2 describes the technique, in the ultrasound imaging, to switch displaying a screen for measurement and a monitor screen in order to ensure the accuracy of diagnosis as well as shorten the diagnostic time required, which leaves problems that the use of the technique is limited to the medical institution equipped with these devices and limited for the ultrasound imaging.

The technique described in Patent Document 3 is construed as the technique to connect such as one specialized hospital equipped with an advanced image pickup equipment, e.g., an MR apparatus, and a plurality of medical institutions without the advanced image pickup equipment via the communication link, to transmit the picked-up image to the remote medical institution so that a residing physician specialized in interpreting the medical image and physicians in the plurality of medical institutions cooperate to view the image in real time to perform identification, investigation, or diagnosis. However, since not only the patients to be diagnosed but also the specialized physicians gather to such as the one specialized hospital, it is possible that the patients and medical staff are forced to wait and influenced by the schedule of the specialized hospital, causing degradation in efficiency.

The technique described in Patent Document 4 is construed as the technique to transmit unprocessed medical image data of a patient picked up by a medical image pickup device, process it with a remote terminal to create a medical image for evaluation by an expert, and to enable the remote terminal to control imaging parameters of the medical image pickup device.

However, since it causes labor concentrated on the expert who takes charge of evaluation and a burden increases, and the equipment is occupied which processes the medical image data and creates the medical image at least during evaluation, it makes difficult to perform the efficient medical treatment. In addition, exchanging the data requires both sides to be equipped with the equipments suited for such operations, and in the case where the medical institutions such as a plurality of clinics are supported, all the institutions requires suitable equipments, resulting in the increase in the equipment cost.

### DISCLOSURE OF THE INVENTION

### Problems to be Solved by the Invention

In the present invention, problems to be solved are, upon providing the latest medical technology to a plurality of terminal medical institutions such as a clinic in a remote/underpopulated area: reducing economic burden on the terminal medical institutions as well as on patients for establishing easier access to receive not only one kind of examination but also a plurality of examinations to increase the accuracy of diagnosis and examination; enabling to ease the economic burden upon installation or maintenance of diagnostic examination instruments or medical equipments; reducing the labor or the equipment burden by decreasing the temporal restriction for a specific latest medical institution or an expert; reducing the burden on the patient such as going to a large hospital where specialized facilities are fully equipped with; solving the inconvenience such as the patient being kept waiting for a long time; and enabling the patient to receive close examination or diagnosis easily in a nearby clinic or small hospital.

### Means for Solving the Problems

[Basic Constitution of the Invention]

A main technique of the present invention is: providing a terminal medical institution such as a clinic in a remote/underpopulated area with a plurality of diagnostic examination instruments as terminal equipments performing essential functions; performing diagnosis and examination using these terminal diagnostic examination instruments; transmitting diagnosis and examination data on the terminal side to a complete medical institution such as a large hospital with expert staff via a communication network; carrying out data analysis and processing on the main body side using highly precise data processing means in the complete medical institution; obtaining a close medical examination result at the terminal medical institution by transmitting the processed result to the terminal medical institution via the communication network; and providing the most-advanced diagnosis and examination for a patient.

In addition, the present invention includes, as the development of the technique to connect a terminal equipment in the terminal medical institution with the complete medical institution, the technique for enabling providing medical treatment during maintenance or upon failure of the complete medical institution itself.

The terminal medical institutions can be plural, which are provided with at least, as the terminal equipments, the diagnostic examination instrument, e.g., a test probe, having an essential function capable to be processed at the complete medical institution in order to detect the patient's condition and create required data, data processing means connected with the diagnostic examination instrument to process the data to level required for data transmission, and a communication controller connected with a communication network corresponding to its type to send/receive the data.

The communication network is desired to be the possible highest speed one within an obtainable range, e.g., a broadband network, and a dedicated or general fiber-optic communication network, a wire communication network using such as a telephone line or a power line, a wireless communication network using such as a ground wave or a satellite, and the Internet are used.

[Detailed Constitution of Terminal Medical Institution]

In the terminal medical institution, it is desirable to be provided with, along with the diagnostic examination instrument, the data processing means, and the communication controller: optionally, a terminal medical instrument with these functions; at least a computer such as a personal computer; operation means such as a keyboard or a mouse to operate various instruments; data storage means to store data of the terminal medical instrument or the personal computer; a monitor to visualize image data of the terminal medical equipment or the personal computer; a microphone or a speaker/receiver to input audio into the terminal medical instrument or the personal computer; and a printer to print out medical data or image data.

Particularly, the diagnostic examination instrument is required to be of the grade capable of acquiring and transmitting the essential diagnosis and examination data from/to the complete medical institution. For example, where it is an ultrasonic probe, it is required to have such as an ultrasonic oscillator or receiver to transmit an ultrasonic wave at a required angle, e.g., perpendicular, 60 degrees, or 45 degrees, and a function to convert received data to an electric signal.

Applied as the data processing means would be the one having a function to process the data obtained by the diagnostic examination instrument of the terminal medical institution into communicable information, or having a function to process all the data receivable from the complete medical institution to the data suitable for transmitting to the monitor, audio generating means, or the data storage means.

[Detailed Constitution of Complete Medical Institution]

The complete medical institution includes optionally a large-scale medical institution, a university hospital, or a support medical institution with equivalent functions for support by cooperating with the terminal medical institution, which needs to be provided with: standard medical equipments including the latest medical equipments; a function to complement the diagnostic examination instrument of the terminal medical institution; the data processing means to perform processing (operation, analysis, diagnosis, imaging processing) of the diagnosis and examination data; the communication controller to receive the transmitted data from the communication network and has a function to specify the institution to be supported if needed, as well as to transmit the diagnosis and examination data or the image processed by the data processing means to the terminal medical institution; an access controller which is actuated upon reception of the transmitted data to initiate the data processing; the data storage means to perform storing of various data if needed; various types of the diagnostic examination instruments with the functions at least more than those at the cooperating terminal medical institution to be supported.

The complete medical institution is connected with a mobile terminal which has desirably a function equivalent to that of the terminal equipment of the terminal medical institution, if needed.

The communication controller of the complete medical institution is provided with a main body/terminal switching connector for establishing connection with the mobile terminal between a main part of the terminal instrument used for such as diagnosis of the patient (a person to be diagnosed) and the main body side (main body of the device) which performs such as data processing, image processing, or data transmission.

This main body/terminal switching connector is provided with means, e.g., a cable or a connector, for establishing connection among the main part of the above-mentioned terminal device, the main body side (main body of the device), and the mobile terminal.

In addition, in order to support the medical treatment, diagnosis, and examination of the terminal medical institution, there is included a medical instrument manufacturer, a support institution, or even a stationary institution with experts responsive to such as the diagnostic examination instrument, wherein the medical instrument manufacturer or the support institution, for supporting the complete medical institution when the failure occurs, is organically connected via such as the communication network.

[Detailed Constitution of Other Support Institutions]

Other support institutions are desirably arranged along with the complete medical institution.

They are connected via the communication network or directly with wires (such as a cable), when a data processing request or support request from the terminal medical institution occurs and the data analysis/processing function of the complete medical institution is degraded for a certain reason (e.g., the latest medical instrument is in use, the expert is absent temporarily) so that it cannot respond to the support request from the terminal medical institution, or when the medical instrument of the complete medical institution itself breaks down and needs repair, or needs maintenance.

For example, while the other support institutions have the terminal equipments similar or equivalent to those of the terminal medical institution or more, it is desirable to be provided with at least the operation means, the data storage means, the monitor, audio input means, the audio generating means, and the data processing means, wherein the above-mentioned medical instrument manufacturer or various examination institutions are included.

[Detailed Constitution of Mobile Terminal]

The complete medical institution is connected with the mobile terminal which offers support when one of their diagnostic examination instruments breaks down, or moves to the place of the complete medical institution for performing diagnosis and examination instead.

The mobile terminal is provided with at least the diagnostic examination instrument having the functions more than equivalent to those of the diagnostic examination instrument of the terminal medical institution and equivalent to those of the diagnostic examination instrument of the complete medical institution, wherein the mobile terminal includes that mounted on a vehicle, a railroad vehicle, a vessel, an aircraft, or a helicopter.

As for the communication means, it is desirable to be provided with that equivalent to the communication controller described relating to the terminal medical institution or the complete medical institution.

### EFFECTS OF THE INVENTION

(1) By arranging a terminal equipment of a diagnostic examination instrument essential to a terminal medical institution to diagnose and examine a patient at the terminal medical institution, and receiving support by a complete medical institution if needed regarding the details of a diagnosis and examination result, it becomes easy to provide the latest medical treatment to a medical institution or a clinic in a remote/underpopulated area which is likely to be left by progress in the medical technology.

(2) By the above, the equipment cost of the terminal medical institution can be reduced, while the economic burden for the patient can also be reduced.

(3) By performing a plurality of examinations at the terminal medical institution, highly accurate medical treatment can be easily provided as compared with the case where a simple home medical treatment is performed.

(4) By arranging the terminal equipment of the essential diagnostic examination instrument, a plurality of examinations can be made easier to receive at the terminal medical institution, so that accuracy of diagnosis/examination can be increased.

(5) By simplifying the equipment of the terminal medical institution, maintenance can be easily carried out.

(6) By transmitting diagnosis and examination data of the terminal medical institution to the complete medical institution for analysis and processing, reliable diagnosis, examination, and medical treatment can be carried out.

(7) It becomes easy to distribute the diagnosis and examination to the terminal medical institution, so that concentration of the patients to such as a university hospital can be reduced and solving the inconvenience such as the patient being kept waiting for a long time can be solved.

(8) By adopting the technique of connecting the terminal medical institution and the complete medical institution by a communication network, maintenance at the complete medical institution itself can be simplified and interruption of the medical treatment upon failure of the medical instrument can be reduced.

(9) By arranging data storage means, a monitor for visualizing image data, a microphone, a speaker/receiver, a printer or the like in the terminal medical institution, medical information with voice can be effectively exchanged with the complete medical institution or provided to the patient.

(10) By arranging a communication controller in the complete medical institution to initiate processing of transmitted data from the terminal medical institution or to specify the institution to be supported, detailed response can be carried out corresponding to a request from the terminal medical institution even when the complete medical institution is crowded.

(11) By utilizing part of the medical equipments of the complete medical institution at the terminal medical institution, an operating ratio and efficiency of such as the latest medical equipments and specialized staff of the complete medical institution is increased, so that early recovery and cost reduction of investment costs for such as equipments can be planned.

(12) By dividing the medical equipments and mounting only the essential diagnostic examination instruments in such as an ambulance, types of the diagnostic examination instruments which may be mounted are increased and a medical support range is broadened, so that accurate emergency medical care can be carried out.

### BRIEF DESCRIPTION OF THE DRAWINGS

FIG. 1 is a block diagram illustrating a basic constitution of a first embodiment of a support network system of a medical institution according to the present invention;

FIG. 2 is a flow chart illustrating a support condition by the basic constitution of FIG. 1;

FIG. 3 is a block diagram illustrating a second embodiment of the support network system of a medical institution;

FIG. 4 is a block diagram illustrating a third embodiment of the support network system of the medical institution;

FIG. 5 is a block diagram illustrating a fourth embodiment of the support network system of the medical institution;

FIG. 6 is a block diagram illustrating a fifth embodiment of the support network system of the medical institution;

FIG. 7 is a flow chart illustrating plural processing conditions upon diagnosis and examination by the medical institution;

FIG. 8 is a flow chart of a maintenance method of a terminal (terminal equipment) in a terminal medical institution;

FIG. 9 is a block diagram illustrating a usage condition of a mobile terminal when a malfunction occurs at a complete medical institution;

FIG. 10 is a block diagram illustrating a condition of testing whether the malfunction occurs at a diagnostic examination instrument in FIG. 9;

FIG. 11 is a block diagram illustrating a condition of testing whether the malfunction occurs at a main body in FIG. 9;

FIG. 12 is a block diagram illustrating a backup condition at the main body side in FIG. 9; and

FIG. 13 is a block diagram illustrating a maintenance condition of the diagnostic examination instrument by a support institution in FIG. 9.

### DESCRIPTION OF REFERENCE NUMERALS

A: terminal medical institution, B: complete medical institution, C: communication network, D: support institution, E: mobile terminal, 10: terminal equipment, 11: diagnostic examination instrument, 11a: endoscope, 11b: suction bottle, 11c: examination subsidiary means, 11d: electrode, 11e: connector, 11f: luminescent means, 12: operation means, 13: data storage means, 14: monitor, 15: voice input means, 16: voice generating means, 17: data processing means 18: switching connector, 20: communication controller, 20a: A/D conversion means (analog/digital conversion means), 20b: photoelectric conversion means, 30: terminal equipment, 31: diagnostic examination instrument, 32: operation means, 33: data storage means, 34: monitor, 35: voice input means, 36: voice generating means, 37: data processing means, 40: communication controller, 40a: A/D conversion means (analog/digital conversion means), 40b: photoelectric conversion means, 40c: main body/terminal switching connector, 50: access controller, 61: diagnostic examination instrument, 62: vehicle, m: ultrasonic echograph terminal, n: ultrasonic echocardiograph terminal, o: endoscope (upper part) terminal, p: endoscope (lower part) terminal, q: electroencephalograph terminal, r: electrocardiograph terminal, s: MRI/MRA terminal, t: X-ray CT terminal, u: blood tester terminal, v: protein diagnostic instrument terminal, M: ultrasonic echograph main body, N: ultrasonic echocardiograph main body, O: endoscope (upper part) main body, P: endoscope (lower part) main body, Q: electroencephalograph main body, R: electrocardiograph main body, S: MRI/MRA main body, T: X-ray CT main body, U: blood tester main body, V: protein diagnostic instrument main body, Z: main body side (main body of device).

### Best Modes for Carrying Out the Invention

The present invention enables, by arranging a terminal equipment of a diagnostic examination instrument essential to a terminal medical institution so that it cooperates with a complete medical institution via a communication network, to provide the most-advanced medical treatment even when a patient is diagnosed and examined at the terminal medical institution, and to reduce economic burden and time burden on the terminal medical institution or the patient, as well as to enhance the functionality of the complete medical institution itself, while achieving the best medical treatment with currently-obtainable medical technology, communication technology or the like.

### FIRST EMBODIMENT

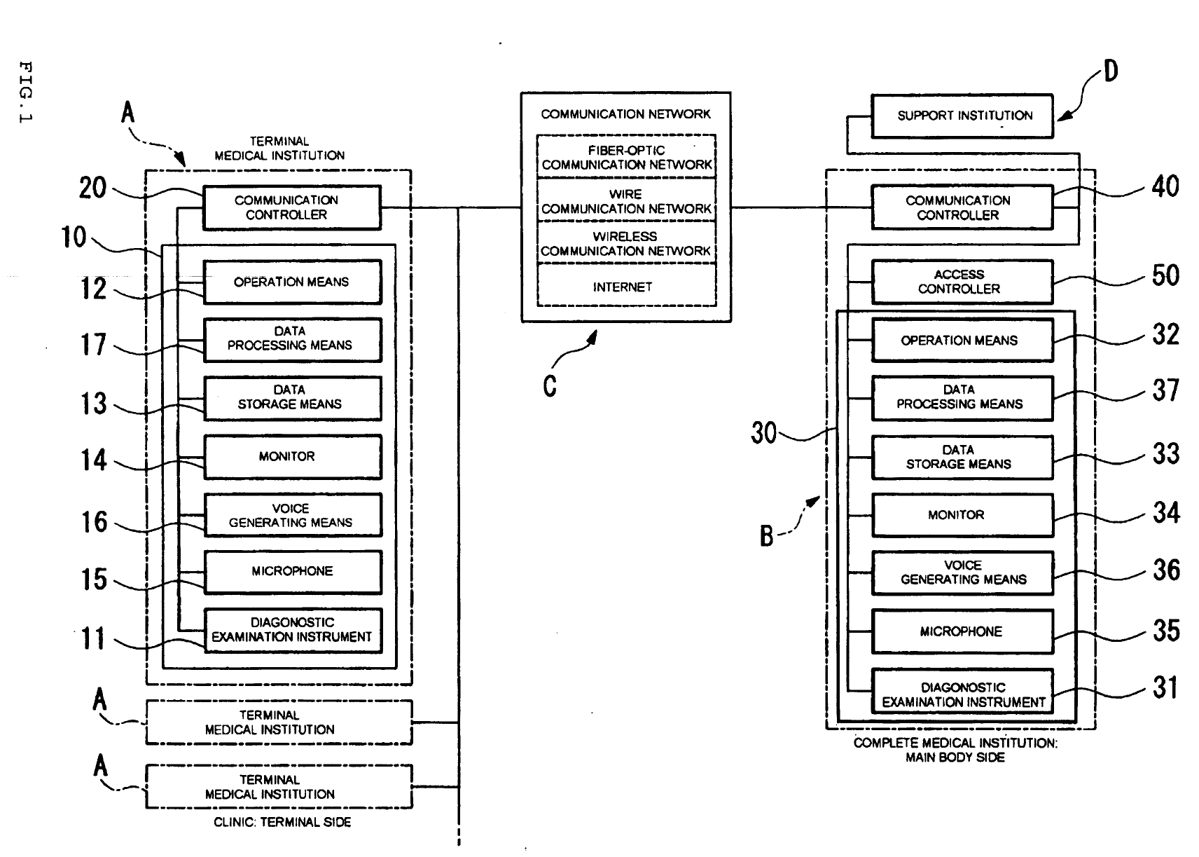

FIG. 1 illustrates a basic constitution of a support network system of a medical institution according to the present invention, wherein a terminal medical institution A and a complete medical institution B are connected via a communication network C. In addition the complete medical institution B is connected with a support institution D if needed.

The terminal medical institution A is such as a clinic in a remote/underpopulated area or a medium or small size hospital where the latest medical equipments or the like are insufficient, which has, as illustrated in FIG. 1, a terminal equipment 10 and a communication controller 20.

While the terminal medical institution A is not limited only to one place but it can be a plurality of places, the terminal equipment 10 is desirably provided with at least a diagnostic examination instrument 11, e.g. a test probe, for detecting the patient's condition, a computer function such as a personal computer, operation means 12 such as a keyboard or a mouse for operating various instruments, data storage means 13 for storing various types of data, a monitor 14 for displaying such as a static image, a video, various data, or text information, voice input means 15 such as a microphone for inputting voice or the like, voice generating means 16 such as a speaker or a receiver connected to the data storage means 13 or the communication network C, data processing means 17 for processing the various data obtained by the diagnostic examination instrument 11 or sent from the complete medical-institution B, and a printer for printing out such as medical data or image data.

For further explanation regarding the diagnostic examination instrument 11, it is required to have a function of the grade capable of acquiring the essential diagnosis and examination data which the complete medical institution B can analyze.

For example, where it is an ultrasonic probe, it is at least required to have such as an ultrasonic oscillator or receiver capable of transmitting an ultrasonic wave at a required angle, e.g., perpendicular, 60 degrees, or 45 degrees, as well as a function to convert received data to an electric signal.

In this case, although it does not eliminate having a function capable of process the data and display an image on the monitor 14 only with the terminal equipment 10, it should have the minimum function of the grade which can be analyzed in the complete medical institution B.

For further explanation regarding the data storage means 13, it contains the various data obtained with the diagnostic examination instrument 11, information related to the terminal medical institution A, the complete medical institution B, and the communication network C, information of the patient, or information analyzed in the complete medical institution B and transmitted to the terminal medical institution A, and which is provided with a hard disk or other storage media for storing these data.

For further explanation regarding the data processing means 17, applied is that having a function to process the data obtained by the diagnostic examination instrument 11 of the terminal medical institution A into communicable information, or a function to process all the data receivable from the complete medical institution B to the data suitable for transmitting to the monitor 14, audio generating means 16, or the data storage means 13.

For further explanation regarding the communication controller 20, it is suitable which lies between the terminal equipment 10 and the communication network C to send/receive communicable data suited for the type of the communication network C.

The complete medical institution B is a university hospital or a large-scale medical institution equipped with the most-advanced medical equipments in such as an urban area, wherein, as illustrated in FIG. 1, it is suitable to be provided with equipments or human resources for cooperating with and supporting the terminal medical institution A, and it has a terminal equipment 30, a communication controller 40, and an access controller 50.

Applied as the terminal equipment 30 is that not only provided with a function more than equivalent to the terminal equipment 10 of a plurality of the terminal medical institutions A, but also has the latest function which complement them, and further provided with a diagnostic examination instrument 31, operation means 32, data storage means 33, a monitor 34, voice input means 35, voice generating means 36, and data processing means 37, which have functions superior to the respective means in the terminal medical institution A, i.e., the diagnostic examination instrument 11, the operation means 12, the data storage means 13, the monitor 14, the voice input means 15, the voice generating means 16, and the data processing means 17.

The communication controller 40 usually actuates the terminal equipment 30 and the data processing means 37 when there are a support request or transmitted data from the terminal medical institution A via the communication network C, to perform necessary data processing, that is, processing (such as analysis, diagnosis, visualization) of medical data by making full use of the most-advanced technology.

When the terminal medical institution A is not capable of processing data (when the data analysis processing capability is insufficient) for the reasons, e.g., the diagnostic examination instrument 31 of the terminal equipment 30 is actuated and the data processing thereof is under operation, a selection is carried out whether to reserve the medical data in the data storage means 33 temporarily or to process the medical data using the support institution D.

In addition, upon completion of processing the medical data in the complete medical institution B, processing is performed to transmit the result to the terminal medical institution A.

It is desirable for the communication network (communication means) C to be high speed communication means within an obtainable range among the terminal medical institution A and the complete medical institutions B, wherein it uses at least one of, preferably two or more of, a fiber-optic communication network, a wire communication network using such as a cable or an electric wiring, a wireless communication network including a satellite communication, and the Internet, for example.

[Ultrasonic Diagnosis and Examination]

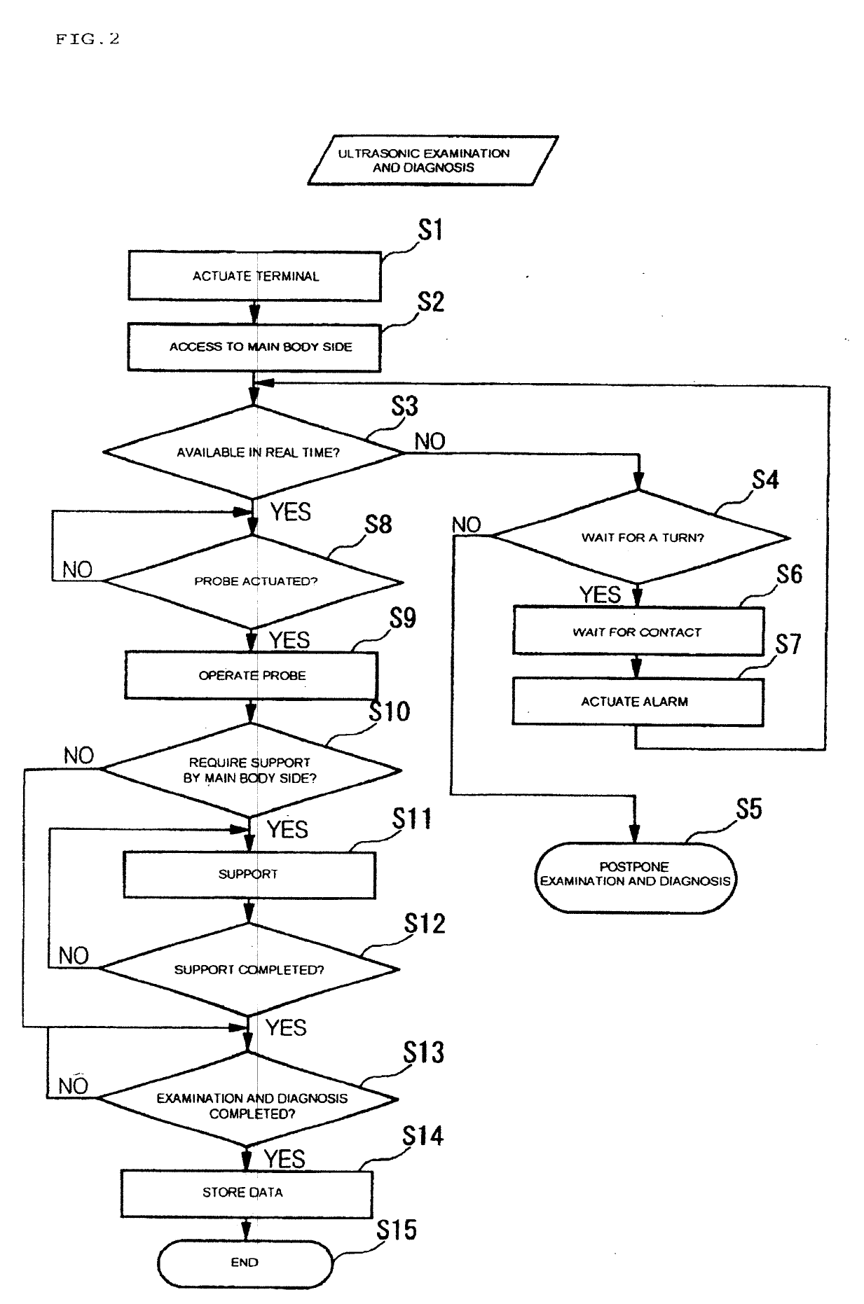

Based on FIG. 2, a support condition by the basic constitution of FIG. 1 will be described.

Hereafter, respective steps will be described individually based on a flow chart.

“S1”: In the terminal medical institution A of FIG. 1, perform an ultrasonic diagnosis and examination of the patient, and actuate the diagnostic examination instrument 11 (terminal: in this case, an ultrasonic examination instrument) of the terminal equipment 10.

“S2”: When the support by the complete medical institution B is required, access the complete medical institution B using the communication network C.

“S3”: Determine whether the main body (such as the data processing means 37) of the complete medical institution B is available in real time.

The determination is usually performed by determining whether the data processing means 37 is not in use and, if it is in use, whether the interruption of processing is possible.

When it is not available in real time (i.e., “no”), the process proceeds to “S4”, and when it is available (i.e., “yes”), it proceeds to “S8.”

“S4”: When not available, select whether or not to wait for a turn. When it does not wait for a turn (i.e., “no”), the process proceeds to “S5,” and when it waits for a turn (i.e., “yes”), it proceeds to “S6.”

“S5”: In the case to interrupt the support by the complete medical institution B, and to cancel the diagnosis and examination requiring the support, postpone the diagnosis and examination.

“S6”: In the case to wait for a turn, stand by and wait for a contact from the complete medical institution B.

“S7”: When contacted by the complete medical institution B, actuate an alarm (the voice generating means 16).

In “S3”, determine whether it is available in real time again, and if it is “yes,” the process proceeds to “S8.”

“S8”: Confirm whether the diagnostic examination instrument 11 (test probe) is actuated. When it is not actuated (i.e., “no”), re-actuate the diagnostic examination instrument 11, and when it is actuated (i.e., “yes”), the process proceeds to “S9.”

“S9”: Operate the diagnostic examination instrument 11 and initiate the diagnosis and examination of the patient.

“S10”: Determine whether the support by the main body side (e.g., analysis by the complete medical institution B) is required from such as the image obtained from the diagnosis and examination.

When the support is required (i.e., “yes”), the process proceeds to S11, and when the result which does not require the support is obtained (i.e., “no”), it proceeds to S13.

“S11”: The support by the complete medical institution B includes not only the analysis of the transmitted medical data but also the voice by a specialized physician for example.

“S12”: Determine whether or not to terminate the support. When it is “yes,” the process proceeds to “S13,” and when it is “no,” it proceeds to “S11” and the support is continued.

“S13”: Determine whether the diagnosis and examination in the terminal medical institution A is completed. When it is “yes,” the process proceeds to “S14,” and when it is “no,” the diagnosis and examination is terminated and the determination thereof is performed.

“S14”: Perform storing of such as the medical data upon completion of the diagnosis and examination.

“S15”: By storing the data, a series of processings of the ultrasonic examination is completed.

### SECOND EMBODIMENT

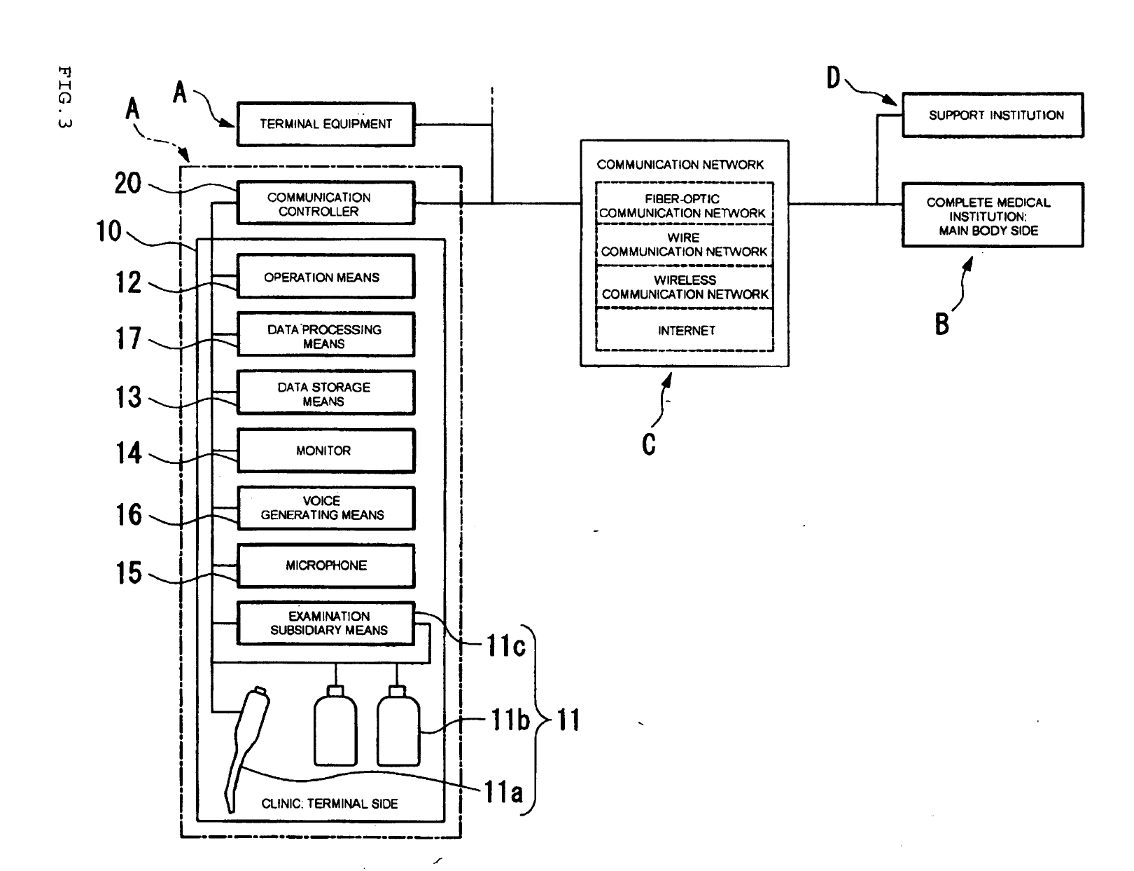

FIG. 3 illustrates a second embodiment of the support network system of the medical institution.

While the complete medical institution B, the communication network C, and the support institution D are with the same constitutions as those of the first embodiment in FIG. 1, there is applied, as the diagnostic examination instrument 11 arranged in the terminal equipment 10 of the terminal medical institution A, the one provided an endoscope 11a to be inserted into the patient's body for comprehending an internal situation and optionally carrying out ablation or depletion, a suction bottle 11b for sucking in such as blood and cleaning fluid using vacuum pressure, and an examination subsidiary means 11c for assisting operation and actuation with these instruments.

For other parts and functions, they are equivalent to those in FIG. 1 and the explanation thereof is omitted. In this case as well, the functions of the diagnostic examination instrument 11 can be kept to the essential level and the video or the like is transmitted to the complete medical institution B for receiving the support of analyzing the video, as well as the support regarding such as the operation of the endoscope 11a or the suction bottle 11b.

### THIRD EMBODIMENT

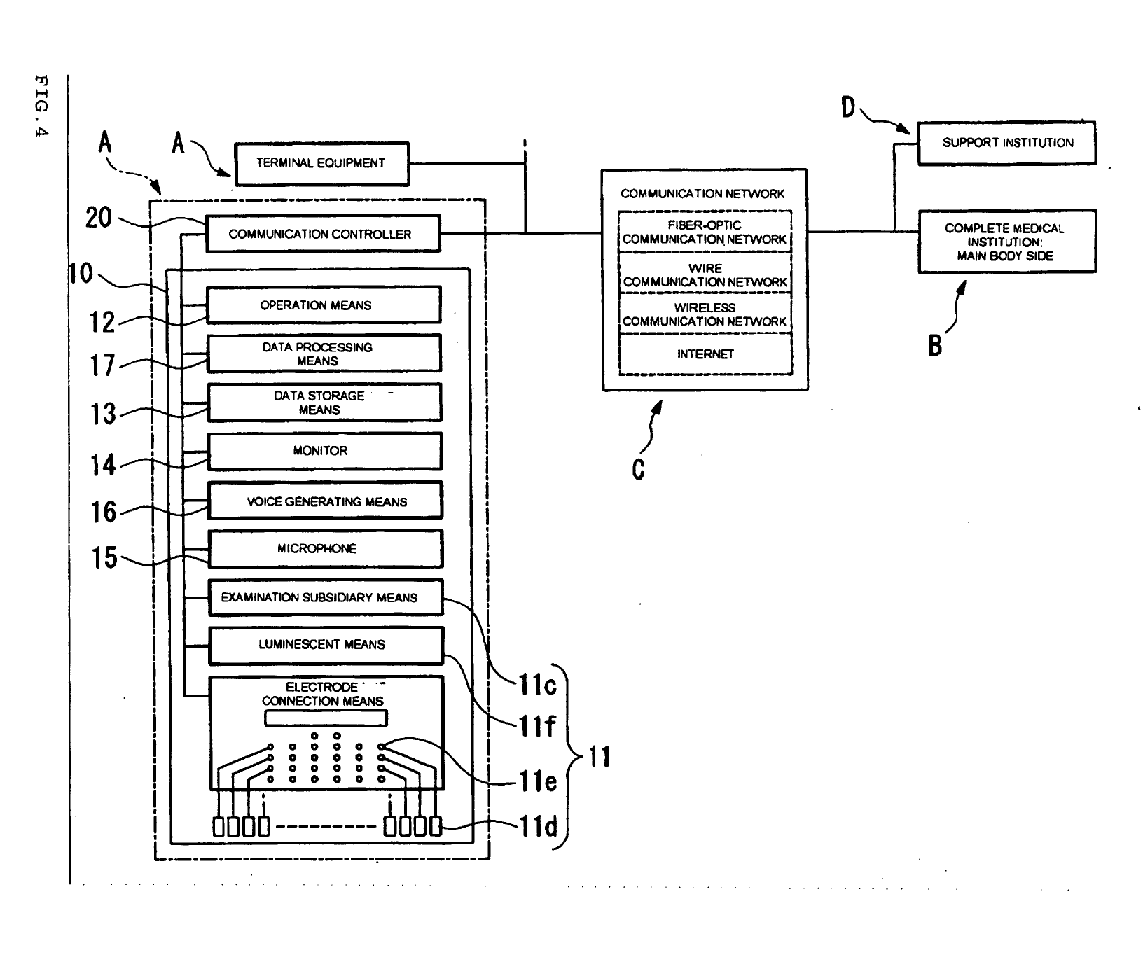

FIG. 4 illustrates a third embodiment of the support network system of the medical institution.

The complete medical institution B, the communication network C, and the support institution D are with the same constitutions as those of the embodiment in FIG. 1

A brain-wave examination terminal is applied as the diagnostic examination instrument 11 arranged in the terminal equipment 10 of the terminal medical institution A.

The diagnostic examination instrument 11 is provided with a plurality of electrodes 11d which are attached to an examination site of the patient, a connector 11e to connect the respective electrodes 11d, and luminescent means 11f for generating such as a pulse light if needed, wherein operations thereof are assisted by examination subsidiary means 11c.

For other parts and functions, they are equivalent to those in FIG. 1 and the explanation thereof is omitted.

In the case of performing the brain-wave examination as well, the functions of the diagnostic examination instrument 11 can be kept to the essential level and the video or the like is transmitted to the complete medical institution B for receiving the support of analyzing the video, as well as the support regarding such as the operation of the diagnostic examination instrument 11.

### FOURTH EMBODIMENT

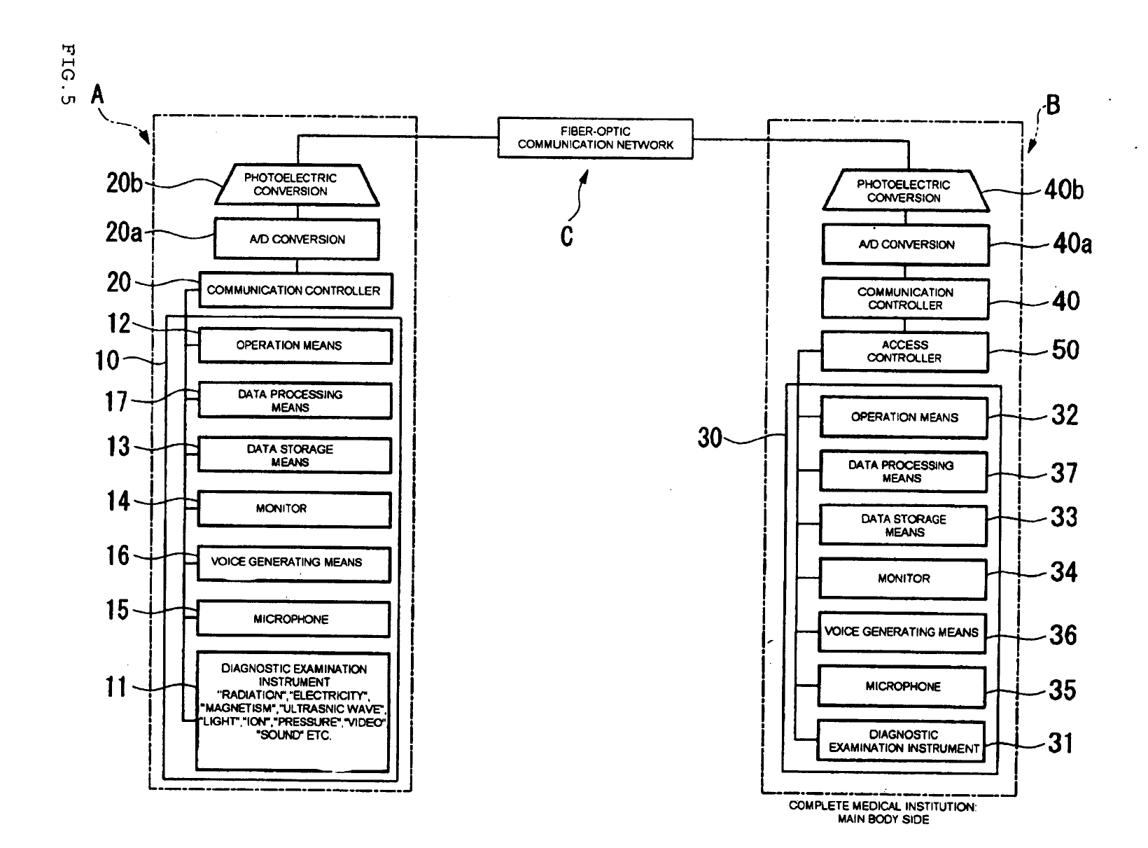

FIG. 5 illustrates a fourth embodiment of the support network system of the medical institution.

The diagnostic examination instrument 11 arranged in the terminal equipment 10 of the terminal medical institution A includes a probe or the like utilizing various sensors which use, for example, “radiation,” “electricity,” “magnetism,” “ultrasonic wave,” “light,” “ion,” “pressure,” “video,” “sound,” and other sensors, which are not taken up in the first through third embodiments.

In addition, as subsidiary functions of the communication controllers 20, 40, when the communication network C uses an optical fiber, A/D conversion means (analog/digital conversion means) 20a, 40a or photoelectric conversion means 20b, 40b are provided in order to make suitable of the data transmission among the terminal medical institution A, the complete medical institution B, and the communication network C.

### FIFTH EMBODIMENT

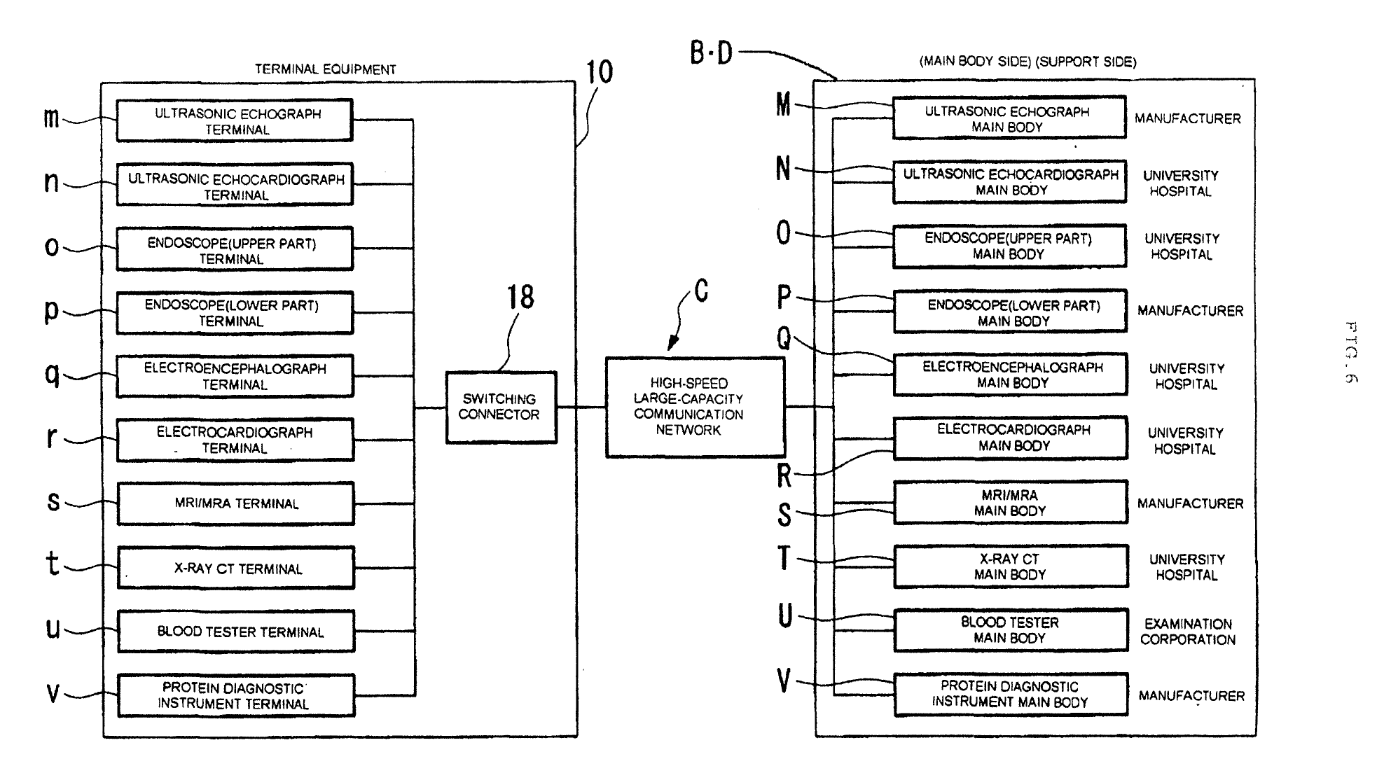

FIG. 6 illustrates a fifth embodiment 5 of the support network system of the medical institution.

In the terminal equipment 10 of the terminal medical institution A, there is provided, as the essential and plurally selected terminals (e.g., a probe part), an ultrasonic echograph terminal m, an ultrasonic echocardiograph terminal n, an endoscope (upper part) terminal o, an endoscope (lower part) terminal p, an electroencephalograph terminal q, an electrocardiograph terminal r, an MRI/MRA terminal s, an X-ray CT terminal t, a blood tester terminal u, and a protein diagnostic instrument terminal v.

In addition to the functions described above, the terminal equipment 10 is provided with a switching connector 18 for switching the respective terminals m through v as appropriate to connect to the communication network C and to transmit such as the medical data.

In addition, in the complete medical institution B or the support institution D as the main body side, there is provided an ultrasonic echograph main body M, an ultrasonic echocardiograph main body N, an endoscope (upper part) main body O, an endoscope (lower part) main body P, an electroencephalograph main body Q, an electrocardiograph main body R, an MRI/MRA main body S, an X-ray CT main body T, a blood tester main body U, and a protein diagnostic instrument main body V, for performing the data processing of the respective terminals m through v as well as the data processing of the terminal of the complete medical institution itself.

As adscripted in FIG. 6, support examples considered to be suitable for the complete medical institution B or the support institution D are, for example, a manufacturer of such as the medical instruments or the computers, the university hospital, or an examination corporation.

### SIXTH EMBODIMENT

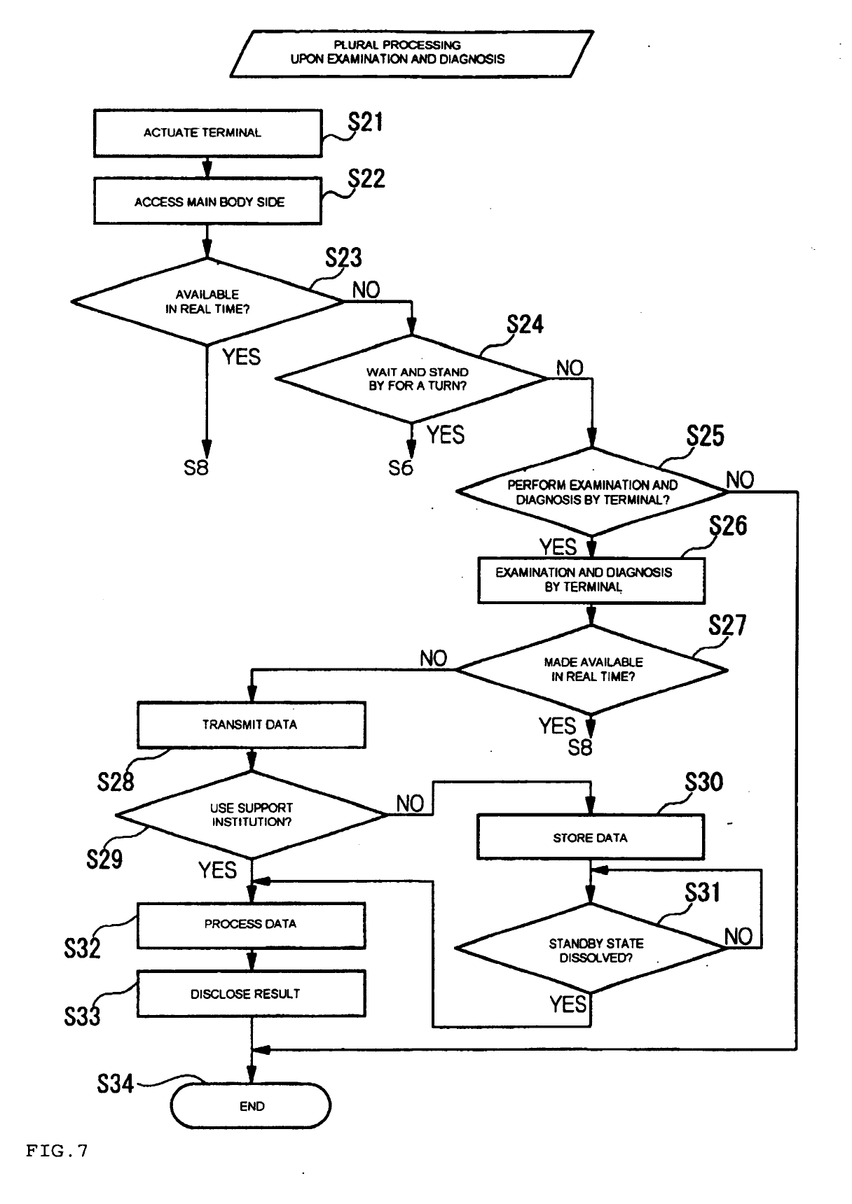

FIG. 7 shows a flow chart illustrating plural processing conditions upon diagnosis and examination by the medical institution.

Hereafter, respective steps will be described individually based on the flow chart.

“S21”: Initiate the diagnosis and examination in the terminal medical institution.

“S22”: Access the complete medical institution using the communication network.

“S23”: Determine whether the complete medical institution is available in real time.

When it is available in real time (i.e., “yes”), the process proceeds to “S8” shown in FIG. 2 for further processing, and when it is not available in real time (i.e., “no”), it proceeds to “S24.”

“S24”: Determine whether to wait for a turn of the support by the complete medical institution. When it waits for a turn (i.e., “yes”), the process proceeds to “S6” shown in FIG. 2 for further processing, and when it does not wait for a turn (i.e., “no”), it proceeds to “S25.”

“S25”: While keeping the state without the support by the complete medical institution, determine whether to perform the diagnosis and examination using the terminal. When it is “yes,” the process proceeds to “S26,” and when it is “no,” it proceeds to “S34” and interrupts or terminates the diagnosis.

“S26”: Perform the diagnosis and examination using the terminal.

“S27”: During use or on termination of the terminal, determine whether the complete medical institution is made available in real time.

When it is available in real time (i.e., “yes”), the process proceeds to “S8” shown in FIG. 2 for further processing, and when it is not available in real time (i.e., “no”), it proceeds to “S28.”

“S28”: Transmit the medical data obtained at the terminal to the support institution via the communication network.

“S29”: Determine whether to use the support institution instead (or whether it is available) which backs up the complete medical institution.

When it is not used (i.e., “no”), the process proceeds to S30, and when it is used (i.e., “yes”), it proceeds to S32.

“S30”: Store the medical data temporarily in the data storage means of the terminal medical institution (see such as FIG. 1).

“S31”: Determine whether a standby state is dissolved and the support by the complete medical institution is made available. When it is “no,” the determination is repeated, and when it is made available (i.e., “yes”), the process proceeds to S32.

“S32”: Perform the processing required for the medical data of the terminal medical institution by the complete medical institution and the support institution.

“S33”: Transmit the processed result of the medical data to the terminal medical institution via the communication network, and carry out such as displaying required for the result.

“S34”: Sufficient explanation is provided for the patient and the process is completed.

### SEVENTH EMBODIMENT

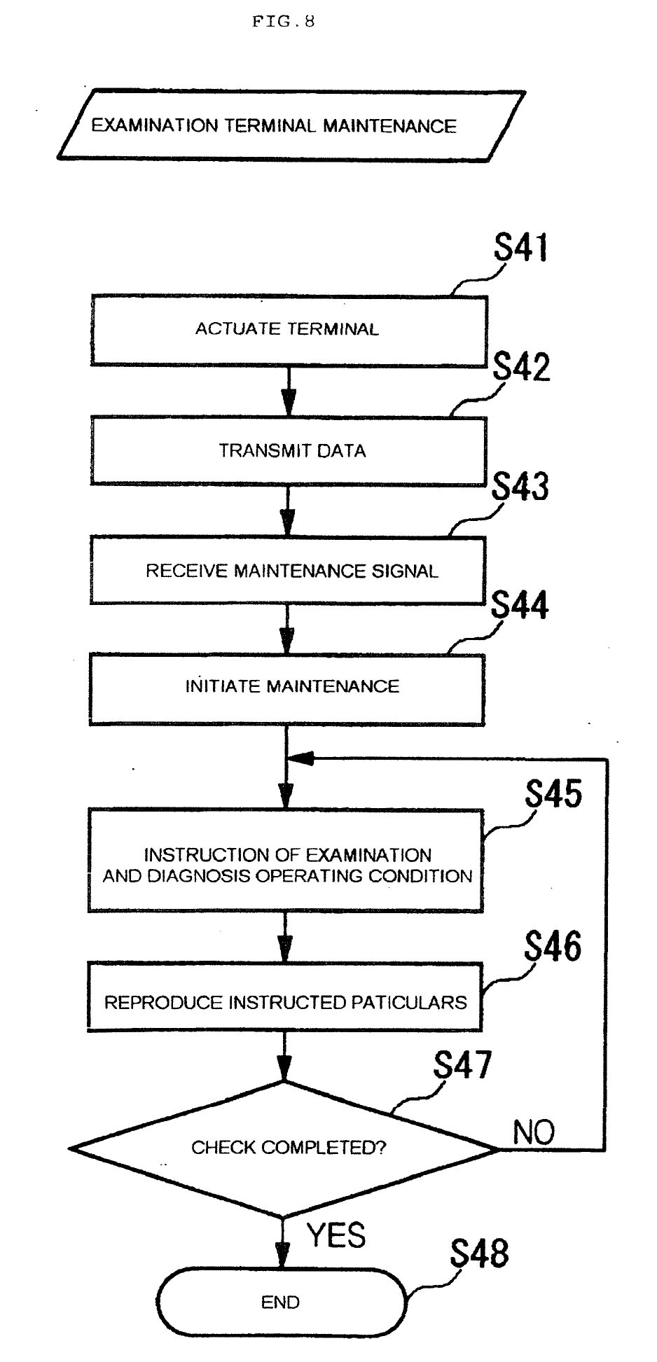

FIG. 8 illustrates a flow chart of a maintenance method of the terminal (terminal equipment) in the terminal medical institution.

While the terminal equipment of the terminal medical institution utilizes the latest diagnosis and examination technology by processing the data with the data processing means 37 of the complete medical institution (see such as FIG. 1), it can perform a remote maintenance by entrusting the maintenance on the complete medical institution.

Hereafter, respective steps will be described individually based on the flow chart.

“S41”: Actuate such as the target terminal (diagnostic examination instrument) at the terminal medical institution to perform the maintenance.

“S42”: Access the complete medical institution to transmit the medical data.

“S43”: Receive the medical data at the complete medical institution.

“S44”: Initiate the maintenance upon reception of the medical data.

“S45”: Confirm that the terminal equipment is operating normally by such as analyzing the medical data from the terminal medical institution, and give instructions regarding the diagnosis and examination method, the operation or actuation conditions of the terminal equipment from the complete medical institution if needed.

“S46”: Perform operation or actuation of the terminal equipment at the terminal medical institution based on the instructions.

“S47”: Confirm the terminal equipment of the terminal medical institution is normal, and select whether or not to terminate the maintenance.

When it cannot be terminated (i.e., “no”), the process proceeds to S45, and when the maintenance is terminated (i.e., “yes”), it proceeds to S48.

“S48”: By cooperation of the main body side of the complete medical institution and the terminal equipment (terminal side) of the terminal medical institution, the maintenance is completed.

### EIGHTH EMBODIMENT

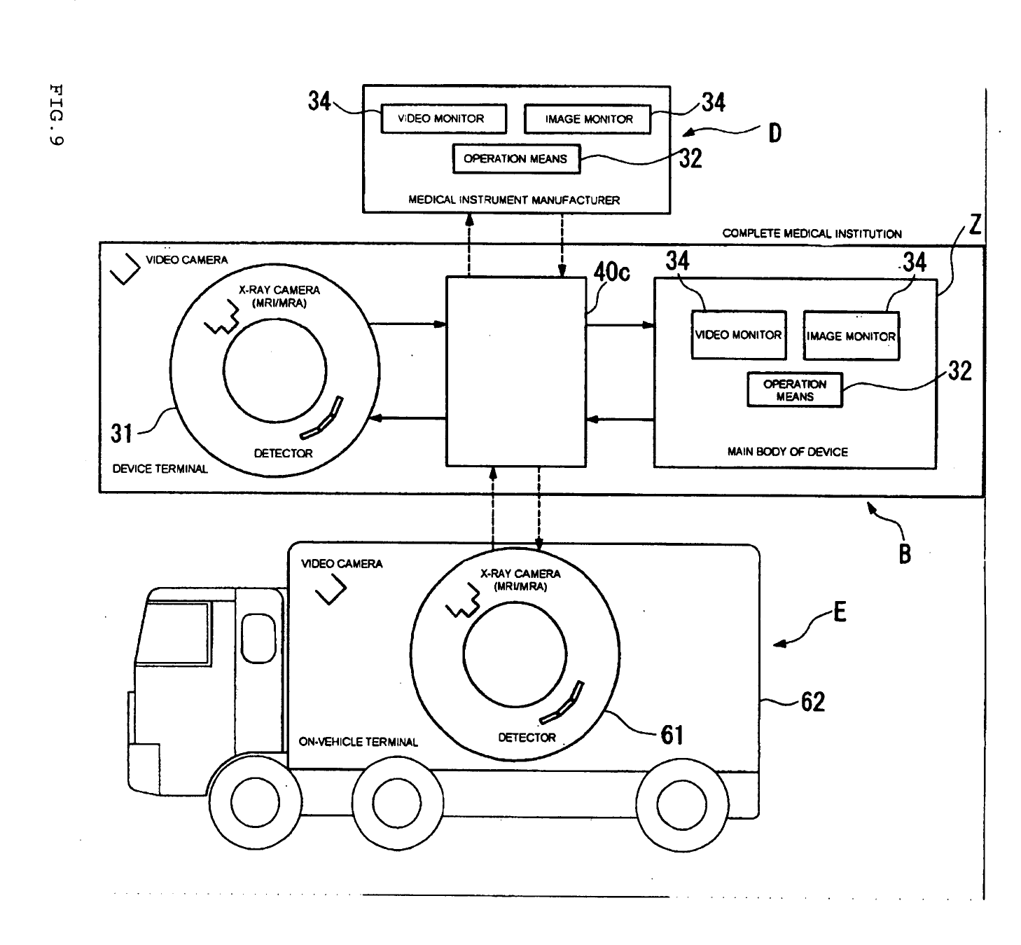

FIGS. 9 through 12 illustrate the solutions when such as the failure occurs in the complete medical institution.

When the failure occurs or it is repaired in the complete medical institution B, the support institution D and a mobile terminal E are used in order to reduce interruption of the most-advanced medical treatment.

As illustrated in FIG. 9, the complete medical institution B is provided with a main body/terminal switching connector 40c constituting a part of the communication controller 40 (shown in such as FIG. 1) between the diagnostic examination instrument 31 and the main body side (main body of the device).

While the support institution D and the mobile terminal E are connected to the main part/terminal switching connector 40c by the communication network, such as an electric cable or the optical fiber, the diagnostic examination instrument 31 and the main body side Z are usually connected by the electric cable.

The main body side Z has the functions such as the operation means 32, the data storage means 33, the monitor 34, the voice input means 35, the voice generating means 36, and the data processing means 37, shown in such as FIG. 1.

The support institution D is such as the medical instrument manufacturer, provided with the functions such as the operation means 32 equivalent to that of the main body side of the complete medical institution B, the monitor 34, and the data processing means 37 illustrated in such as FIG. 1.

The mobile terminal E is provided with the diagnostic examination instrument 31 which the complete medical institution B originally possesses, or at least a diagnostic examination instrument 61, shown as such as MRI/IRA in FIG. 9, having a function of the diagnostic examination instrument 11 of the terminal equipment 10, and is brought to be movable by mounting the diagnostic examination instrument 61 on a vehicle 62.

FIG. 9 illustrates the condition where, in the case that the failure occurs or is likely to occur at the complete medical institution B, the mobile terminal E is moved to the complete medical institution B and stood by, while a test is carried out to determine whether the diagnostic examination instrument 31 or the main body side Z is normal.

As shown by a solid-line arrow in FIG. 9, the diagnostic examination instrument 31 and the main body side Z are used to test whether a malfunction occurs. When the malfunction is acknowledged, it determines that either of the diagnostic examination instrument 31 or the main body side Z breaks down.

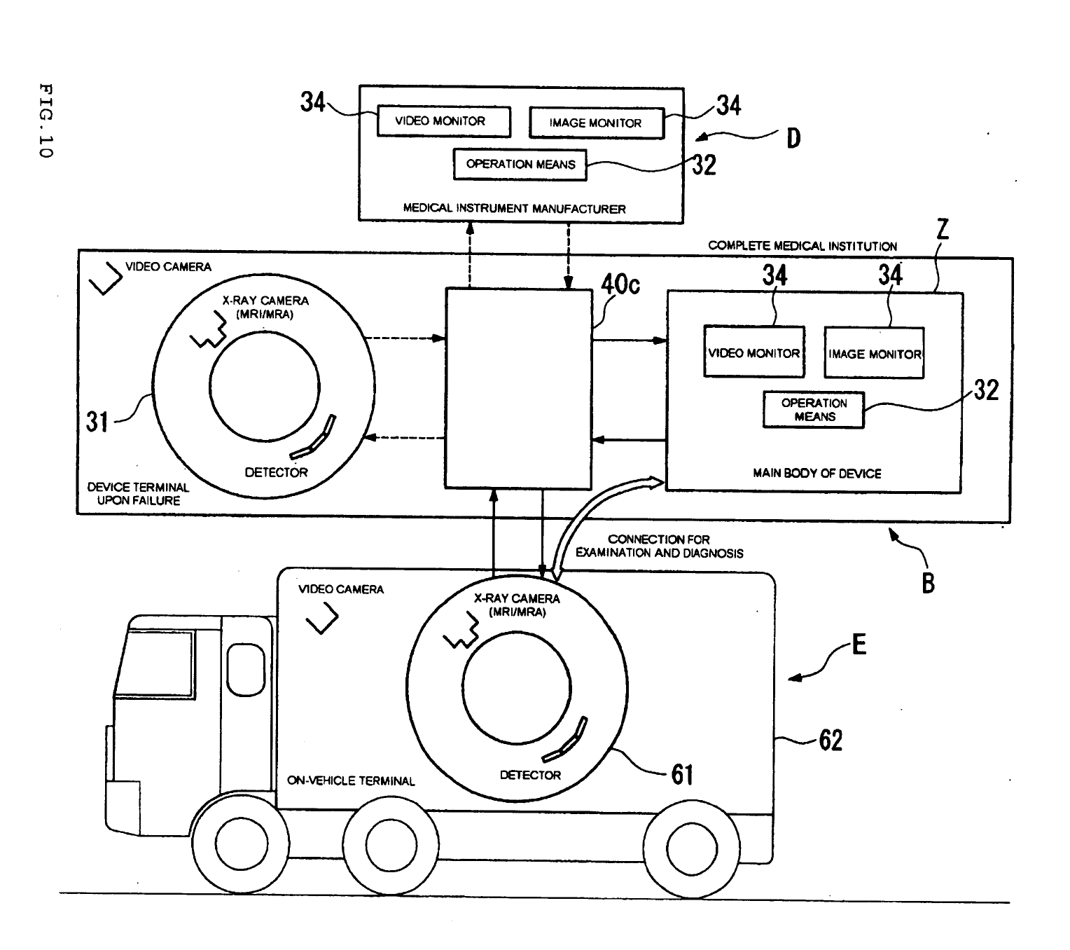

FIG. 10 illustrates the condition where the test is performed to determine whether the malfunction occurs at the diagnostic examination instrument 31.

As shown by a solid-line arrow, the main body side Z and the mobile terminal E are connected via the main part/terminal switching connector 40c to confirm whether the combination operates normally.

When the malfunction is not acknowledged in this combination and the malfunction is acknowledged in either of the diagnostic examination instrument 31 or the main body side Z, it determines that the diagnostic examination instrument 31 malfunctions and, while repairing and checking it, the diagnosis and examination can be performed by guiding the patient to the mobile terminal E.

The diagnosis and examination are performed by the diagnosis examination instrument 61 while utilizing the most-advanced main body side Z, as shown by a white arrow.

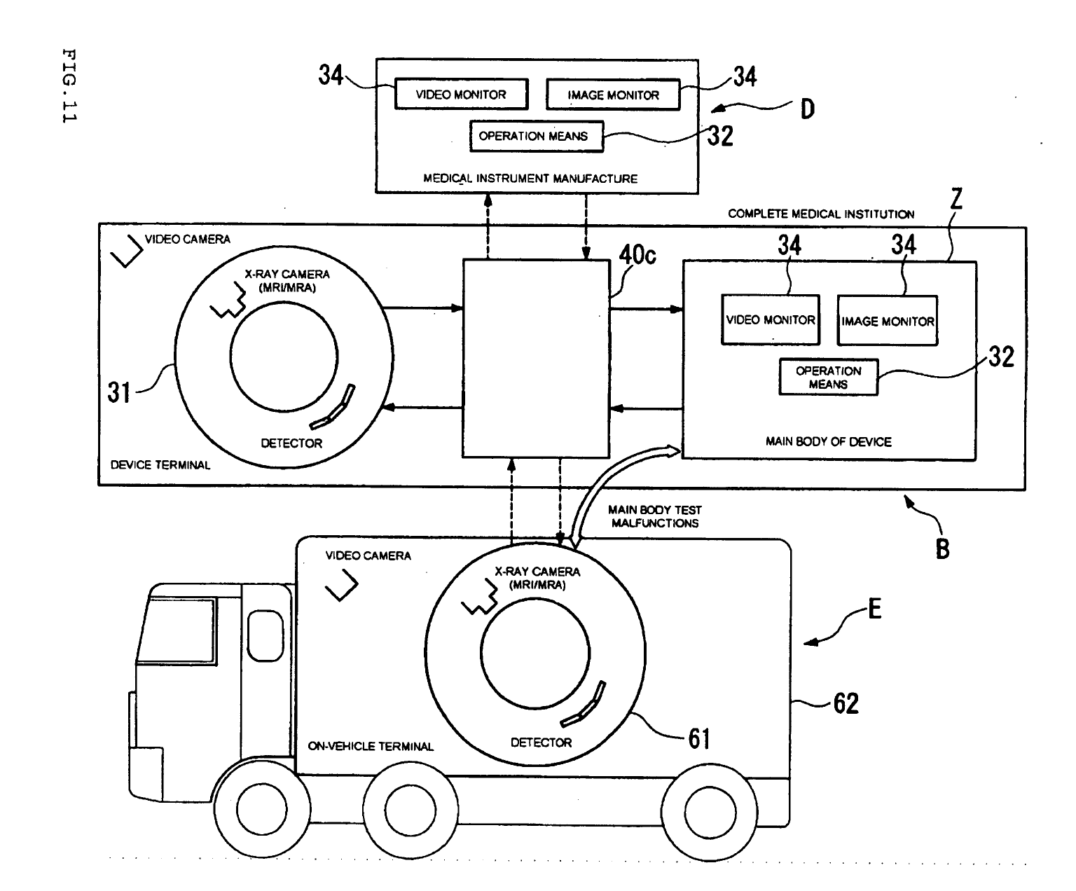

FIG. 11 illustrates the condition where the test is performed to determine whether the malfunction occurs at the main body side Z.

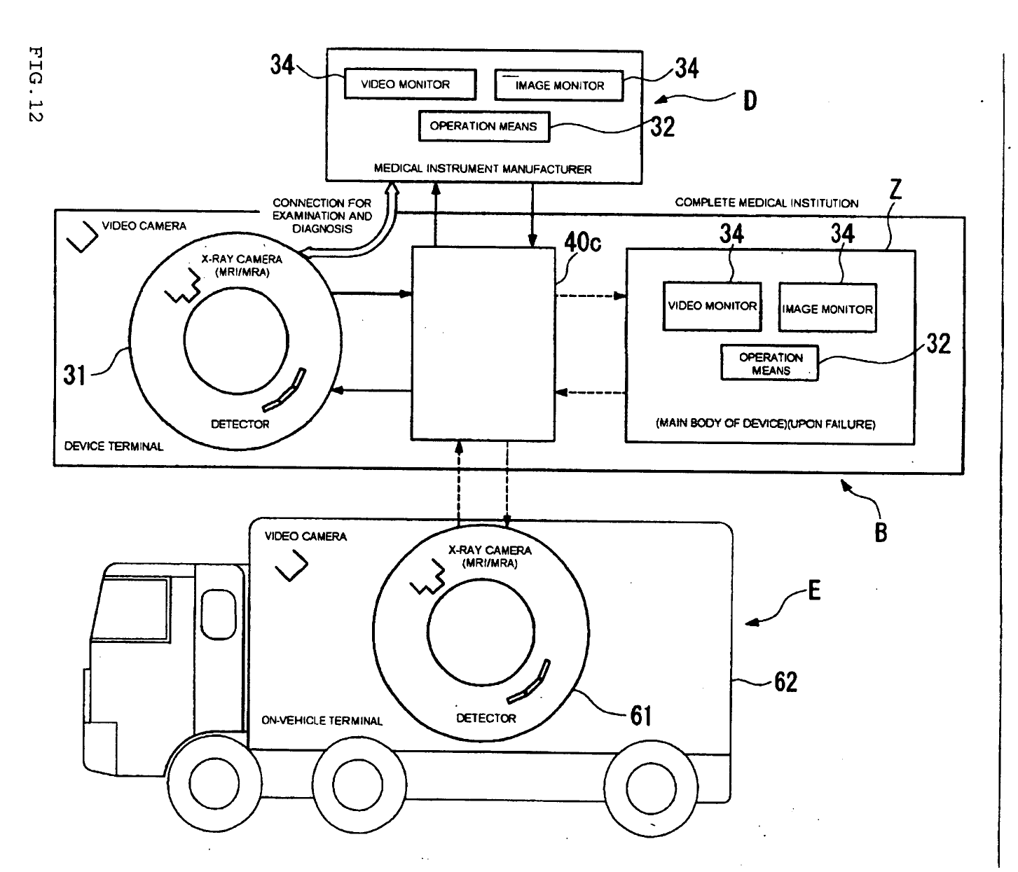

When the malfunction occurs on the usual condition where the diagnostic examination instrument 31 and the main body side Z are connected, it determines that the diagnostic examination instrument 31 is doubtful and the diagnosis and examination are performed using the mobile terminal E. However, the malfunction is still acknowledged even after switched to the mobile terminal E as shown by a solid-line arrow in FIG. 11, it determines that the main body side Z malfunctions as shown by a white arrow in FIG. 11 and the backup is carried out as illustrated in FIG. 12.

FIG. 12 illustrates a backup condition at the main body side Z of the complete medical institution B.

When the main body side Z malfunctions breaks down and the grade of the failure is limited to such as the data processing means 37, while the operation means 32, the data storage means 33, the monitor 34, the voice input means 35, the voice generating means 36, and the communication controller 40 are normal, the diagnosis and examination are performed using the support institution D and the diagnostic examination instrument 31, along with performing repair, replacement, check or the like of the main body side Z, as shown by solid-line and white arrows in FIG. 12.

Even in this diagnosis and examination, the most-advanced medical treatment level is not degraded fundamentally.

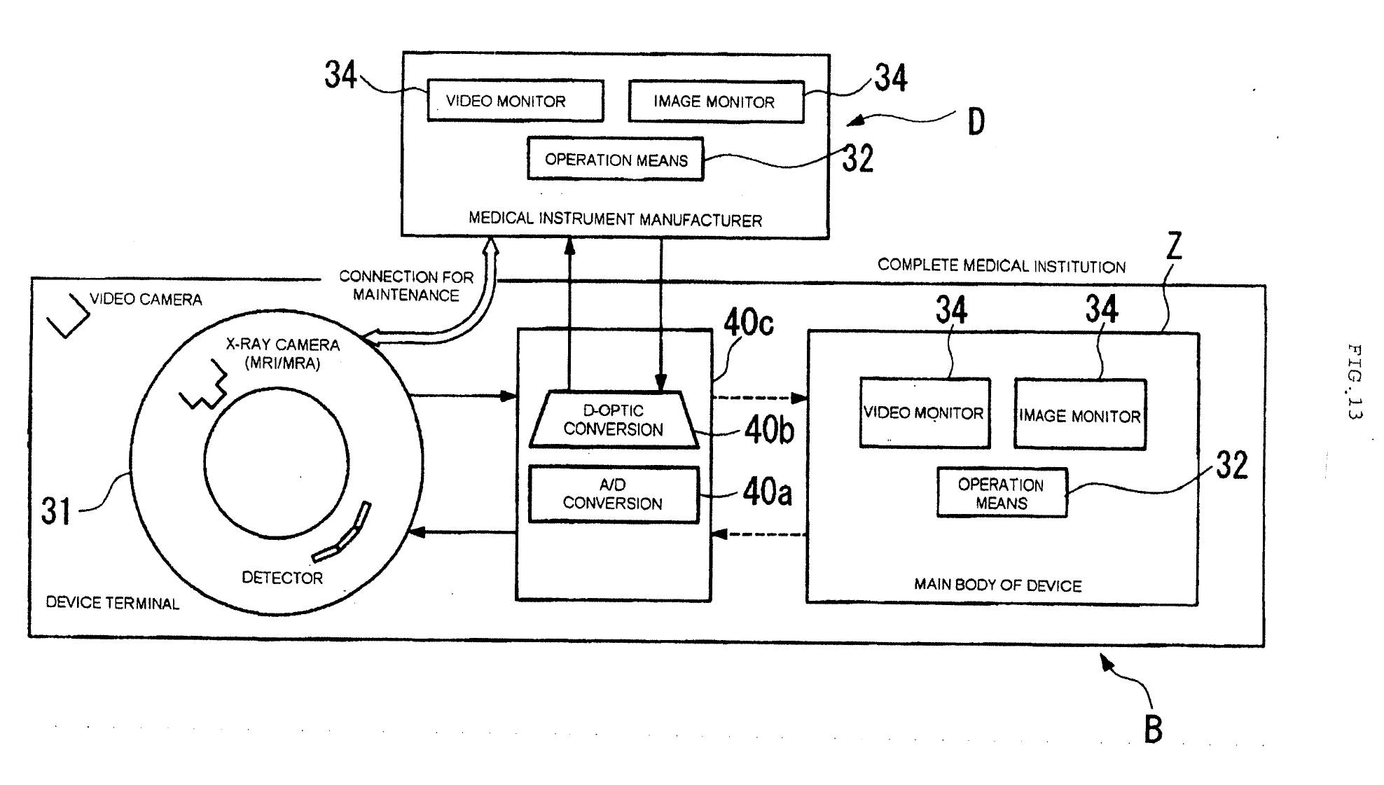

FIG. 13 illustrates a maintenance condition of the diagnostic examination instrument 31 by the support institution D.

When it is obvious that the failure is caused by the main body side Z, it is possible to perform the diagnosis and examination using the support institution D and the diagnostic examination instrument 31 as shown by solid-line and white arrows, without using the mobile terminal E illustrated in FIGS. 9 through 12.

After performing repair, replacement, check or the like of a malfunctioning part of the main body side Z to bring it back to normal, the diagnostic examination instrument 31 and the main body side Z are connected to carry out the test, as shown by a dashed-line arrow, and if it is acknowledged that the performance is not different from that performed using the support institution D and the normal condition is restored, the support institution D is disconnected and it may return to the diagnosis and examination condition with only the complete medical institution B.

### INDUSTRIAL APPLICABILITY

(1) In the near future for example, when there is improvement in the medical equipments of the complete medical institution or improvement in the automated diagnosis technique by such as the computer regarding the medical information stored in a database, it is still possible to provide the most-advanced medical treatment for the patient of the diagnosis and examination by the terminal medical institution.

(2) Even when there is improvement in performance of the diagnostic examination instrument, it is possible to control the equipment investment of the terminal medical institution and to carry out the support by the complete medical institution continuously.

(3) By modifying medical service systems or legislations, it is applicable to the medical support when an emergency case arises in an ambulance, an emergency helicopter, other transportation facilities, or public facilities.

1. A support method of a medical institution, characterized by providing the latest medical technology for a terminal medical institution, wherein a diagnosis and an examination are provided for a patient of the terminal medical institution by:
arranging a terminal equipment having a diagnostic examination instrument essential to the terminal medical institution;
performing the diagnosis and examination of the patient by the terminal equipment at the terminal medical institution;
transmitting diagnosis and examination data of the terminal equipment to a complete medical institution via a communication network;
analyzing and processing the data at the complete medical institution; and
transmitting the processed result to the terminal medical institution via the communication network.

2. The support method of the medical institution according to claim 1, wherein data analysis/processing is performed by transferring diagnosis and examination data to other support institution equivalent to the complete medical institution while a data analysis/processing function of the complete medical institution is degraded.

3. A support network of a medical institution providing the latest medical technology for a terminal medical institution, comprising:
a terminal equipment having a diagnostic examination instrument essentially arranged in the terminal medical institutions;
a communication network for transmitting diagnosis and examination data of the terminal equipment; and
a complete medical institution for analyzing and processing the diagnosis and examination data transmitted and for transmitting the processed result to the terminal medical institution via the communication network.

4. The support network of the medical institution according to claim 3, wherein the communication network is connected with a support institution having a function equivalent to the data processing/analyzing function of the complete medical institutions.

5. The support network of the medical institution according to claim 3, wherein the complete medical institution is provided with a mobile terminal having a function equivalent to that of the terminal medical institutions.

6. The support network of the medical institution according to claim 3, wherein the terminal medical institution comprises terminal medical institutions distributed in a plurality of places.

7. The support network of the medical institution according to claim 3, wherein the terminal medical institution comprises:
a diagnostic examination instrument for detecting the condition of the patient;
data processing means connected to the diagnostic examination instrument for processing the data; and
a communication controller or connecting to the communication network for transmission.

8. The support network of the medical institution according to claim 3, wherein the terminal medical institution comprises a monitor for an image/video transmitted from the complete medical institution.

9. The support network of the medical institution according to claim 3, wherein the complete medical institution comprises a communication controller for receiving the transmitted data from the communication network, having an function to specify the institution to be supported.

10. The support network of the medical institution according to claim 3, wherein the complete medical institution comprises an access controller to be actuated upon reception of the transmitted data for initiating the data processing.

11. The support network of the medical institution according to claim 3, wherein the communication network is connected with a support institution having a function equivalent to the data processing/analyzing function of the complete medical institution and wherein the complete medical institution is provided with a mobile terminal having a function equivalent to that of the terminal medical institution.

12. The support network of the medical institution according to claim 3, wherein the communication network is connected with a support institution having a function equivalent to the data processing/analyzing function of the complete medical institution and wherein the terminal medical institution comprises terminal medical institutions distributed in a plurality of places.

13. The support network of the medical institution according to claim 3, wherein the communication network is connected with a support institution having a function equivalent to the data processing/analyzing function of the complete medical institution and wherein the terminal medical institution comprises:
a diagnostic examination instrument for detecting the condition of the patient;
data processing means connected to the diagnostic examination instrument for processing the data; and
a communication controller for connecting to the communication network for transmission.

14. The support network of the medical institution according to claim 3, wherein the complete medical institution is provided with a mobile terminal having a function equivalent to that of the terminal medical institution and wherein the terminal medical institution comprises:
a diagnostic examination instrument for detecting the condition of the patient;
data processing means connected to the diagnostic examination instrument for processing the data; and
a communication controller for connecting to the communication network for transmission.

15. The support network of the medical institution according to claim 3, wherein the complete medical institution is provided with a mobile terminal having a function equivalent to that of the terminal medical institution and wherein the terminal medical institution comprises a monitor for an image/video transmitted from the complete medical institution.

16. The support network of the medical institution according to claim 3, wherein the complete medical institution comprises a communication controller for receiving the transmitted data from the communication network, having an function to specify the institution to be supported, and wherein the terminal medical institution comprises:
a diagnostic examination instrument for detecting the condition of the patient;
data processing means connected to the diagnostic examination instrument for processing the data; and
a communication controller for connecting to the communication network for transmission.

17. The support network of the medical institution according to claim 3, wherein the terminal medical institution comprises a monitor for an image/video transmitted from the complete medical institution and wherein the complete medical institution comprises a communication controller for receiving the transmitted data from the communication network, having an function to specify the institution to be supported.

18. The support network of the medical institution according to claim 3, wherein the communication network is connected with a support institution having a function equivalent to the data processing/analyzing function of the complete medical institution and wherein the complete medical institution comprises an access controller to be actuated upon reception of the transmitted data for initiating the data processing.

19. The support network of the medical institution according to claim 3, wherein the terminal medical institution comprises:
a diagnostic examination instrument for detecting the condition of the patient;
data processing means connected to the diagnostic examination instrument for processing the data; and
a communication controller for connecting to the communication network for transmission, and
wherein the complete medical institution comprises an access controller to be actuated upon reception of the transmitted data for initiating the data processing.

20. The support network of the medical institution according to claim 3, wherein the terminal medical institution comprises a monitor for an image/video transmitted from the complete medical institution and wherein the complete medical institution comprises an access controller to be actuated upon reception of the transmitted data for initiating the data processing.
